(19) United States
(12) Patent Application Publication
(10) Pub. No.: US 2025/0049759 A1
(43) Pub. Date: Feb. 13, 2025

(54) PHARMACEUTICAL PREPARATION FOR ASSISTING IN PROMOTING SLEEP AND PREPARATION METHOD THEREOF

(71) Applicant: Changde Jizhi Biological Technology Co., Ltd, Changde (CN)

(72) Inventor: Xinqiao HE, Changde (CN)

(21) Appl. No.: 18/230,749

(22) Filed: Aug. 7, 2023

(51) Int. Cl. A61K 31/4045; A61K 9/00; A61K 31/30; A61K 33/24; A61K 47/22; A61P 25/20

(52) U.S. Cl. CPC A61K 31/4045; A61K 9/0053; A61K 31/30; A61K 33/24; A61K 47/22; A61P 25/20

(57) ABSTRACT

A pharmaceutical preparation for assisting in promoting sleep and a preparation method thereof are provided, and the pharmaceutical preparation is prepared from the following raw materials: small molecular group water, melatonin, sodium copper chlorophyllin, tea polyphenol and nano-titanium dioxide. Pharmacodynamic studies show that the pharmaceutical preparation has a good effect of assisting in promoting sleep.

### STATEMENT REGARDING PRIOR DISCLOSURES BY THE INVENTOR OR A JOINT INVENTOR

Chinese Publication No. CN114869908A dated Aug. 9, 2022 is a grace period inventor-originated disclosure which qualifies as an exception under 35 U.S.C. 102 (b) (1).

### TECHNICAL FIELD

The disclosure relates to the field of biomedicine technologies, and more particularly to a pharmaceutical preparation for assisting in promoting sleep and a preparation method thereof.

### BACKGROUND

Insomnia, mainly refers to manifestations of various sleep disorders such as long duration and difficulty from a start of sleep to complete sleep, a continuous poor quality sleep state, easy awakening during sleep, short sleep time, often waking up before predetermined time, and difficulty in falling asleep after waking up. Traditional Chinese medicine has various names for the insomnia, such as “sleepless”, “not sleeping”, “not lying down” and “restless eyes”. Modern medical research has shown that sleep-wake occurs alternately under the regulation of central neurotransmitter factors such as the hypothalamus-pituitary-adrenal axis, dopamine, 5-hydroxytryptamine, neuropeptides, acetylcholine, and γ-aminobutyric acid, and hormone regulator factors such as melatonin and prostaglandin D2 (PGD2) and cytokines such as interleukins-1. interleukin-6, and tumor necrosis factors. If these factors are abnormal, it may cause the insomnia.

At present, the social competition is fierce, the living environment is under great pressure, the crowd is highly concentrated and tense, and a combination of various factors leads to an increase in the incidence of insomnia year by year. According to literature reports, the overall prevalence rate of insomnia in people over 18 years old was 14.5%, while the prevalence rates of males and females were 10.3% and 18.1%, respectively, and the prevalence rate increases with age, reaching 23.3% at the age of 65 and above. Such a high prevalence rate seriously affects people's participation in daily work and life, and is seriously detrimental to the development of physical and mental health. Studies have shown that long-term bed rest and psychological factors account for 79.7% and 61.1% of the common causes of insomnia, respectively, while unhealthy lifestyles such as tobacco, alcohol, tea, and coffee, physical factors such as sound, light, and heat, diseases such as perimenopausal comprehensive symptoms and comorbid insomnia, and some special drugs can cause the insomnia.

With the rapid development of modern biomedical technology, benzodiazepine receptor agonists (BZBAs), melatonin receptor agonists, orexin receptor antagonists, antidepressants and other drugs play an important role in the treatment of insomnia. Different types of drugs have different therapeutic effects, methods of use, doses and durations of action in clinical research. Generally speaking, they can shorten the time to fall asleep, prolong and improve the effective sleep time, reduce the number of awakenings, improve sleep quality, and relieve insomnia symptoms, etc. The short-term efficacy of drugs in the treatment of insomnia is certain. Since different drugs have different potential risks such as adverse reactions, addiction, and withdrawal symptoms, the use should be strictly adjusted in accordance with the recommended dose, method of use, and time limit, so as to give full play to the role of drug treatment in taking effect quickly, eliminating symptoms in a short time and avoiding prolonged course of disease. In the treatment methods of Chinese and Western medicine for insomnia, Western medicine mainly uses chemical synthetic drugs to inhibit the sleep center of patients, with fast onset and good effect, and the recent treatment effect is good. From a long-term point of view, there are many adverse reactions such as dizziness the next day, memory loss, drowsiness, influence on cognitive function, drug dependence, and rebound insomnia. Traditional Chinese medicine gives full play to the characteristics and advantages of syndrome differentiation and treatment, mainly using traditional Chinese medicine, and comprehensively applying a variety of methods to adjust the balance of blood and yin-yang in the body. The treatment process is safe, with few side effects, and the effect is outstanding.

For insomnia patients, taking chemical medicines or traditional Chinese medicines can only be obtained by a doctor's prescription. After taking chemical medicines, side effects are large, and traditional Chinese medicines are inconvenient to take. Therefore, it is of great clinical significance to develop a pharmaceutical preparation that is convenient to take and carry.

### SUMMARY

Based on the above reasons, after several creative efforts, the inventor has obtained a pharmaceutical preparation for assisting in promoting sleep. The pharmaceutical preparation is prepared from the following raw materials: small molecular group water, melatonin, sodium copper chlorophyll, tea polyphenol, nano-titanium dioxide; pharmacodynamic research shows that the pharmaceutical preparation of the disclosure has a good effect of assisting in promoting sleep.

The disclosure is implemented through the following technical solution.

A pharmaceutical preparation for assisting in promoting sleep is provided, and the pharmaceutical preparation is prepared from the following raw materials: small molecular group water, melatonin, sodium copper chlorophyllin, tea polyphenol, and nano-titanium dioxide.

In an embodiment, the small molecular group water is also referred to micro cluster water, small cluster water, micro clustered water, or structured water, and within 100 hertz (Hz), a water molecule group is composed of 5-7 water molecules, which is called as the small molecular group.

In an embodiment, in the pharmaceutical preparation, the raw materials in parts by weight are as follows: 100-200 parts by weight of the small molecular group water, 1-3 parts by weight of the melatonin, 15-25 parts by weight of the sodium copper chlorophyllin, 20-40 parts by weight of the tea polyphenol, and 0.1-0.2 parts by weight of the nano-titanium dioxide.

In an embodiment, in the pharmaceutical preparation, the raw materials in parts by weight are as follows: 150 parts by weight of the small molecular group water, 2 parts by weight of the melatonin, 20 parts by weight of the sodium copper chlorophyllin, 30 parts by weight of the tea polyphenol, and 0.15 parts by weight of the nano-titanium dioxide.

In an embodiment, the pharmaceutical preparation is a spray.

In an embodiment, the pharmaceutical preparation is an oral solution.

In an embodiment, the pharmaceutical preparation is used to prepare a medicine for assisting in promoting sleep.

In an embodiment, a preparation method of the pharmaceutical preparation includes:

taking the nano-titanium dioxide, adding small molecular group water of 10-20 times of a weight of the nano-titanium dioxide and stirring evenly; rinsing a stirring rod with small molecular group water of 10-20 times of a weight of the melatonin, adding the melatonin and stirring evenly; then rinsing the stirring rod with small molecular group water of a weight of the sodium copper chlorophyllin, adding the sodium copper chlorophyllin and the tea polyphenol, and then stirring evenly; and rinsing the stirring rod with remaining small molecular group water, and placing the obtained mixture into a ultrasonic oscillator to obtain the pharmaceutical preparation.

TABLE 1
sources of raw materials of the disclosure

| Name of raw material | Purchasing manufacturer | Purchasing date |
|---|---|---|
| small molecular group water | Guangxi Bama Mountain | 2022 Mar. 16 |
| | Spring Natural Drinking Water | |
| | Co., Ltd | |
| melatonin | Weiyipeptide (Guangdong) | 2022 Feb. 23 |
| | Biotechnology Co., Ltd | |
| sodium copper chlorophyllin | Henan Xingding Chemical | 2022 Mar. 1 |
| | Products Co., Ltd | |
| tea polyphenol | Shaanxi Yizhimin Plant | 2022 Mar. 2 |
| | Technology Co., Ltd | |
| nano-titanium dioxide | Bohuasi Nanotechnology | 2022 Mar. 14 |
| | (Ningbo) Co., Ltd | |

The disclosure has beneficial technical effects are as follows:
(1) The tea polyphenol, the sodium copper chlorophyllin, the nano-titanium dioxide and the melatonin are organically combined, and the small molecular group water is added to form an organic whole composition for assisting in promoting sleep.
(2) The preparation process is simple and easy for industrial production.
(3) The pharmaceutical preparation can be made into the oral solution or the spray, which is easy to carry and beneficial to travelers.

### DETAILED DESCRIPTION OF EMBODIMENTS

In order to make the purpose, technical solution, and advantages of the disclosure clearer, the following will provide a further detailed explanation of the disclosure in conjunction with specific implementation methods. It should be understood that these descriptions are only illustrative and not intended to limit the scope of the disclosure. In addition, in the following explanation, the description of well-known structures and techniques has been omitted to avoid unnecessary confusion with the concepts of the disclosure.

### Embodiment 1

Raw materials of a pharmaceutical preparation include: small molecular group water of 150 grams (g), melatonin of 2 g, sodium copper chlorophyllin of 20 g, tea polyphenol of 30 g, and nano-titanium dioxide of 0.15 g.

A preparation method of the pharmaceutical preparation includes the following steps: the nano-titanium dioxide is taken, small molecular group water of 15 times of the weight of the nano-titanium dioxide is added and then stirring evenly; then a stirring rod is rinsed with small molecular group water of 15 times of the weight of the melatonin, the melatonin is added and then stirring evenly; then the stirring rod is rinsed with small molecular group water of the weight of the sodium copper chlorophyllin, then adding the sodium copper chlorophyllin and the tea polyphenol, and stirring evenly; and the stirring rod is rinsed with remaining small molecular group water, and the obtained mixture through the above steps is placed into a ultrasonic oscillator to obtain the pharmaceutical preparation. The pharmaceutical preparation is filled to obtain an oral liquid or prepared according to a conventional preparation method to obtain a spray.

### Embodiment 2

Raw materials of a pharmaceutical preparation include: small molecular group water of 100 g, melatonin of 1 g, sodium copper chlorophyllin of 15 g, tea polyphenol of 20 g, and nano-titanium dioxide of 0.10 g.

A preparation method of the pharmaceutical preparation includes the following steps: the nano-titanium dioxide is taken, small molecular group water of 10 times of the weight of the nano-titanium dioxide is added and then stirring evenly; then a stirring rod is rinsed with small molecular group water of 10 times of the weight of the melatonin, the melatonin is added and then stirring evenly; then the stirring rod is rinsed with small molecular group water of the weight of the sodium copper chlorophyllin, then adding the sodium copper chlorophyllin and the tea polyphenol, and stirring evenly; and the stirring rod is rinsed with remaining small molecular group water, and the obtained mixture through the above steps is placed into a ultrasonic oscillator to obtain the pharmaceutical preparation. The pharmaceutical preparation is filled to obtain an oral liquid or prepared according to a conventional preparation method to obtain a spray.

### Embodiment 3

Raw materials of a pharmaceutical preparation include: small molecular group water of 200 g, melatonin of 3 g, sodium copper chlorophyllin of 25 g, tea polyphenol of 40 g, and nano-titanium dioxide of 0.2 g.

A preparation method of the pharmaceutical preparation includes the following steps: the nano-titanium dioxide is taken, small molecular group water of 20 times of the weight of the nano-titanium dioxide is added and then stirring evenly; then a stirring rod is rinsed with small molecular group water of 20 times of the weight of the melatonin, the melatonin is added and then stirring evenly; then the stirring rod is rinsed with small molecular group water of the weight of the sodium copper chlorophyllin, then adding the sodium copper chlorophyllin and the tea polyphenol, and stirring evenly; and the stirring rod is rinsed with remaining small molecular group water, and the obtained mixture through the above steps is placed into a ultrasonic oscillator to obtain the pharmaceutical preparation. The pharmaceutical preparation is filled to obtain an oral liquid or prepared according to a conventional preparation method to obtain a spray.

### Embodiment 4

Raw materials of a pharmaceutical preparation include: small molecular group water of 120 g, melatonin of 1.5 g, sodium copper chlorophyllin of 18 g, tea polyphenol of 25 g, and nano-titanium dioxide of 0.15 g.

A preparation method of the pharmaceutical preparation includes the following steps: the nano-titanium dioxide is taken, small molecular group water of 14 times of the weight of the nano-titanium dioxide is added and then stirring evenly; then a stirring rod is rinsed with small molecular group water of 14 times of the weight of the melatonin, the melatonin is added and then stirring evenly; then the stirring rod is rinsed with small molecular group water of the weight of the sodium copper chlorophyllin, then adding the sodium copper chlorophyllin and the tea polyphenol, and stirring evenly; and the stirring rod is rinsed with remaining small molecular group water, and the obtained mixture through the above steps is placed into a ultrasonic oscillator to obtain the pharmaceutical preparation. The pharmaceutical preparation is filled to obtain an oral liquid or prepared according to a conventional preparation method to obtain a spray.

### Embodiment 5

Raw materials of a pharmaceutical preparation include: small molecular group water of 180 g, melatonin of 2.5 g, sodium copper chlorophyllin of 22 g, tea polyphenol of 35 g, and nano-titanium dioxide of 0.25 g.

A preparation method of the pharmaceutical preparation includes the following steps: the nano-titanium dioxide is taken, small molecular group water of 18 times of the weight of the nano-titanium dioxide is added and then stirring evenly; then a stirring rod is rinsed with small molecular group water of 18 times of the weight of the melatonin, the melatonin is added and then stirring evenly; then the stirring rod is rinsed with small molecular group water of the weight of the sodium copper chlorophyllin, then adding the sodium copper chlorophyllin and the tea polyphenol, and stirring evenly; and the stirring rod is rinsed with remaining small molecular group water, and the obtained mixture through the above steps is placed into a ultrasonic oscillator to obtain the pharmaceutical preparation. The pharmaceutical preparation is filled to obtain an oral liquid or prepared according to a conventional preparation method to obtain a spray.

### Comparative Example 1

Raw materials of a pharmaceutical preparation include: small molecular group water of 150 g, melatonin of 2 g, tea polyphenol of 30 g, and nano-titanium dioxide of 0.15 g.

A preparation method of the pharmaceutical preparation includes the following steps: the nano-titanium dioxide is taken, small molecular group water of 15 times of the weight of the nano-titanium dioxide is added and then stirring evenly; then a stirring rod is rinsed with small molecular group water of 15 times of the weight of the melatonin, the melatonin is added and then stirring evenly; then the stirring rod is rinsed with small molecular group water of the weight of the tea polyphenol, then adding the tea polyphenol and stirring evenly; and the stirring rod is rinsed with remaining small molecular group water, and the obtained mixture is placed into a ultrasonic oscillator to obtain the pharmaceutical preparation. The pharmaceutical preparation is filled to obtain an oral liquid or prepared according to a conventional preparation method to obtain a spray.

### Comparative Example 2

Raw materials of a pharmaceutical preparation include: small molecular group water of 150 g, melatonin of 2 g, sodium copper chlorophyllin of 20 g, and nano-titanium dioxide of 0.15 g.

A preparation method of the pharmaceutical preparation includes the following steps: the nano-titanium dioxide is taken, small molecular group water of 15 times of the weight of the nano-titanium dioxide is added and then stirring evenly; then a stirring rod is rinsed with small molecular group water of 15 times of the weight of the melatonin, the melatonin is added and then stirring evenly; then the stirring rod is rinsed with small molecular group water of the weight of the sodium copper chlorophyllin, then adding the sodium copper chlorophyllin and stirring evenly; and the stirring rod is rinsed with remaining small molecular group water, and the obtained mixture is placed into a ultrasonic oscillator to obtain the pharmaceutical preparation. The pharmaceutical preparation is filled to obtain an oral liquid or prepared according to a conventional preparation method to obtain a spray.

### Test Example 1

### Effects on Sleep in Mice

### Test Drugs:

Test group 1: diazepam group.
Test group 2: melatonin group.
Test group 3: diazepam+embodiment 1 group.
Test group 4: diazepam+embodiment 4 group.
Test group 5: diazepam+embodiment 5 group.
Test group 6: diazepam+comparative example 1 group.
Test group 7: diazepam+comparative example 2 group.

Note: embodiments and comparative examples were concentrated and dried to obtain test raw materials.

Test animals: Kunming mice, 18-22 g. (specific pathogen free (SPF) grade, provided by the Pharmacology Department of Traditional Chinese Medicine, Beijing University of Chinese Medicine).

Test method: The mice were randomly divided into a control group and a test group. The test group 1: diazepam of 0.75 mg/kg was administrated by gavage once a day for two consecutive weeks. The test group 2: melatonin of 75 mg/kg was administrated by gavage once a day for two consecutive weeks. The test group 3 to test group 7: after administrating diazepam of 0.75 mg/kg by gavage, 3.5 g/kg of the raw material of the embodiment or comparative example is administrated once a day for two consecutive weeks. After 60 minutes of the last intragastric administration, the mice in each group were injected with 50 mg/kg pentobarbital sodium intraperitoneally, and the sleep latency and sleep duration of the mice in each group were recorded with the disappearance of righting reflex more than 1 group as the sleep standard.

Test results: see Table 2.

TABLE 2
effects of pentobarbital sodium of suprathreshold dose
on sleep latency and duration in mice

| | Number | | |
|---|---|---|---|
| | of | Sleep latency | Sleep duration |
| Group | animals | (min) | (min) |
| Control group | 10 | 5.64 ± 0.39 | 22.98 ± 3.71 |
| Test group 1 | 10 | 2.47 ± 0.27** | 126.37 ± 6.17** |
| Test group 2 | 10 | 4.38 ± 0.35 | 64.38 ± 3.70** |
| Test group 3 | 10 | 1.76 ± 0.29**##& | 174.29 ± 6.89**##&ω |
| Test group 4 | 10 | 1.83 ± 0.24**##& | 171.80 ± 7.40**##&ω |
| Test group 5 | 10 | 1.80 ± 0.31**##& | 172.48 ± 7.29**##&ω |
| Test group 6 | 10 | 2.31 ± 0.25* | 119.49 ± 6.06* |
| Test group 7 | 10 | 2.30 ± 0.36** | 117.49 ± 6.29* |

Note:
** P < 0.01, and *P < 0.05 compared with the control group; ##P < 0.01, and #P < 0.05 compared with the test group 2; &&P < 0.01, and &P < 0.05 compared with the test group 1; and ωP < 0.05 compared with the test group 6.

Test conclusion: the above tests show that the raw material composition of the pharmaceutical preparation of the disclosure can promote the medicinal effect of the diazepam, and after removing the tea polyphenol or sodium copper chlorophyllin, the medicinal effect of enhancing diazepam disappears, which fully demonstrates the composition of the disclosure is an organic whole. Although the sodium copper chlorophyllin or tea polyphenol do not have the effect of promoting sleep, they form a whole with the melatonin and nano-titanium dioxide, this whole can promote the pharmacological effect of diazepam, which further promotes fast and sustained sleep time.

The above are only the preferred embodiments of the disclosure. It should be pointed out that for those skilled in the art, without departing from the principles of the disclosure, several improvements and modifications can be made and these should be regarded as the protection scope of the disclosure.

What is claimed is:

1. A pharmaceutical preparation for assisting in promoting sleep, wherein the pharmaceutical preparation is prepared from the following raw materials: small molecular group water, melatonin, sodium copper chlorophyllin, tea polyphenol, and nano-titanium dioxide.

2. The pharmaceutical preparation as claimed in claim 1, wherein the raw materials in parts by weight are as follows:
100-200 parts by weight of the small molecular group water, 1-3 parts by weight of the melatonin, 15-25 parts by weight of the sodium copper chlorophyllin, 20-40 parts by weight of the tea polyphenol, and 0.1-0.2 parts by weight of the nano-titanium dioxide.

3. The pharmaceutical preparation as claimed in claim 1, wherein the raw materials in parts by weight are as follows:
150 parts by weight of the small molecular group water, 2 parts by weight of the melatonin, 20 parts by weight of the sodium copper chlorophyllin, 30 parts by weight of the tea polyphenol, and 0.15 parts by weight of the nano-titanium dioxide.

4. The pharmaceutical preparation as claimed claim 1, wherein the pharmaceutical preparation is a spray.

5. The pharmaceutical preparation as claimed claim 2, wherein the pharmaceutical preparation is a spray.

6. The pharmaceutical preparation as claimed claim 3, wherein the pharmaceutical preparation is a spray.

7. The pharmaceutical preparation as claimed in claim 1, wherein the pharmaceutical preparation is an oral solution.

8. The pharmaceutical preparation as claimed in claim 2, wherein the pharmaceutical preparation is an oral solution.

9. The pharmaceutical preparation as claimed in claim 3, wherein the pharmaceutical preparation is an oral solution.

10. The pharmaceutical preparation as claimed in claim 1, wherein the pharmaceutical preparation is used to prepare a medicine for assisting in promoting sleep.

11. The pharmaceutical preparation as claimed in claim 2, wherein the pharmaceutical preparation is used to prepare a medicine for assisting in promoting sleep.

12. The pharmaceutical preparation as claimed in claim 3, wherein the pharmaceutical preparation is used to prepare a medicine for assisting in promoting sleep.

13. The pharmaceutical preparation as claimed in claim 1, wherein a preparation method of the pharmaceutical preparation comprises:
taking the nano-titanium dioxide, adding small molecular group water of 10-20 times of a weight of the nano-titanium dioxide and stirring evenly; rinsing a stirring rod with small molecular group water of 10-20 times of a weight of the melatonin, adding the melatonin and stirring evenly; then rinsing the stirring rod with small molecular group water of a weight of the sodium copper chlorophyllin, adding the sodium copper chlorophyllin and the tea polyphenol and stirring evenly; and rinsing the stirring rod with remaining small molecular group water to obtain a mixture, and placing the mixture into a ultrasonic oscillator to obtain the pharmaceutical preparation.

14. A use of the pharmaceutical preparation as claimed in claim 1, comprising:
preparing a medicine for assisting in promoting sleep by using the pharmaceutical preparation; and
administrating diazepam with a first dosage and the medicine with a second dosage once a day to a patient with insomnia.

15. The use according to claim 14, wherein the raw materials in parts by weight are as follows:
100-200 parts by weight of the small molecular group water, 1-3 parts by weight of the melatonin, 15-25 parts by weight of the sodium copper chlorophyllin, 20-40 parts by weight of the tea polyphenol, and 0.1-0.2 parts by weight of the nano-titanium dioxide.

16. The use according to claim 14, wherein the raw materials in parts by weight are as follows:
150 parts by weight of the small molecular group water, 2 parts by weight of the melatonin, 20 parts by weight of the sodium copper chlorophyllin, 30 parts by weight of the tea polyphenol, and 0.15 parts by weight of the nano-titanium dioxide.

17. The use according to claim 14, wherein the medicine is a spray or an oral solution.

18. A preparation method of a pharmaceutical preparation, comprising:
dividing water into four parts, adding a first part of the four parts of the water into nano-titanium dioxide and stirring evenly to obtain a first mixture, wherein a weight of the first part of the water is 10-20 times of a weight of the nano-titanium dioxide;
adding a second part of the four parts of the water into the first mixture by rinsing a stirring rod with the second part of the water, then adding melatonin into the first mixture and stirring evenly to obtain a second mixture; wherein a weight of the second part of the water is 10-20 times of a weight of the melatonin;
adding a third part of the four parts of the water into the second mixture by rinsing the stirring rod with the third part of the water, then adding sodium copper chlorophyllin and tea polyphenol into the second mixture, and stirring evenly to obtain a third mixture; wherein a weight of the third part of the water is equal to a weight of the sodium copper chlorophyllin; and
adding a forth part of the four parts the water into the third mixture by rinsing the stirring rod with the forth part of the water to obtain a target mixture, and placing the target mixture into a ultrasonic oscillator to obtain the pharmaceutical preparation.

19. The preparation method according to claim 18, wherein the water is 100-200 parts by weight, the melatonin is 1-3 parts by weight, the sodium copper chlorophyllin is 15-25 parts by weight, the tea polyphenol is 20-40 parts by weight, and the nano-titanium dioxide is 0.1-0.2 parts by weight.

20. The preparation method according to claim 18, wherein the water is 150 parts by weight, the melatonin is 2 parts by weight, the sodium copper chlorophyllin is 20 parts by weight, the tea polyphenol is 30 parts by weight, and the nano-titanium dioxide is 0.15 parts by weight.
